(19) United States
(12) Patent Application Publication
(10) Pub. No.: US 2026/0289745 A1
(43) Pub. Date: Sep. 24, 2026

(54) ARTIFACT MITIGATION IN X-RAY IMAGING

(71) Applicant: Baker Hughes Holdings LLC, Houston, TX (US)

(72) Inventors: David Uribe Gallo, Augsburg (DE); Tobias Neubrand, Hannover (DE)

(21) Appl. No.: 19/088,437

(22) Filed: Mar. 24, 2025

(51) Int. Cl. G06T 5/70; G01N 23/04; G01N 23/083; G06T 5/50

(52) U.S. Cl. CPC G06T 5/70; G01N 23/04; G01N 23/083; G06T 5/50; G01N 2223/204; G01N 2223/401; G06T 2207/10116; G06T 2207/20224

(57) ABSTRACT

A first data set, characterizing multiple primary projections, and a second data set, characterizing multiple secondary projections, are received. Each primary projection includes both primary radiation and secondary radiation. Each secondary projection includes only the secondary radiation. An associated secondary projection is subtracted from each primary projection. A set of corrected images with the secondary radiation removed based on the primary projections is then provided.

### TECHNICAL FIELD

The subject matter described herein relates to X-ray imaging technology.

### BACKGROUND

One of the persistent challenges in X-ray imaging applications is the degradation of image quality due to backscattering electrons in X-ray sources which create a second X-ray signal (secondary X-ray source). The secondary source detected alongside the primary radiation that travels directly from the source's target to the detector. The presence of this scattered radiation in the detected signal results in images that are often blurred or obscured, reducing the clarity and contrast necessary for accurate analysis and interpretation. Conventional X-ray imaging system often requires the integration of physical barriers such as grids or collimators that block secondary rays from reaching the detector, which complicate the imaging setup and reduces the overall intensity of the detected signal.

### SUMMARY

This disclosure relates to artifact mitigation in X-ray imaging.

An example implementation of the subject matter described within this disclosure is a method with the following features. A first data set, characterizing multiple primary projections, and a second data set, characterizing multiple secondary projections, are received. Each primary projection includes both primary radiation and secondary radiation. Each secondary projection includes only the secondary radiation. An associated secondary projection is subtracted from each primary projection. A set of corrected images with the secondary radiation removed based on the primary projections is then provided.

The disclosed method can be implemented in a variety of ways. For example, within a system that includes at least one data processor and a non-transitory memory storing instructions for the processor to perform aspects of the method. Alternatively, or in addition, the method can be in included non-transitory computer readable memory storing the method as instructions which, when executed by at least one data processor forming part of at least one computing system, causes the at least one data processor to perform operations of the method.

Aspects of the example method, that can be combined with the example method alone or in combination with other methods, can include the following. The primary projections are captured using an X-ray tube in a first operational mode, and the secondary projections are captured using the X-ray tube in a second operational mode.

Aspects of the example method, that can be combined with the example method alone or in combination with other methods, can include the following. The second data set is further resampled by interpolating between multiple secondary projections. Resampling the second data set includes the following steps. An angular interval between each secondary projection is determined. The secondary projections are interpolated at the determined angular interval and added back to the second data set.

Aspects of the example method, that can be combined with the example method alone or in combination with other methods, can include the following. The angular interval is determined to range from 0 to 180 degrees.

Aspects of the example method, that can be combined with the example method alone or in combination with other methods, can include the following. Subtracting the associated secondary projection from each primary projection includes following steps. A secondary projection with a highest secondary signal-to-noise ratio is created. A mask is created based on the created secondary projection. The mask is then applied to each secondary projection and a region of interest (ROI) affected by the secondary radiation is then isolated. The isolated ROI is subtracted from each primary projection.

Aspects of the example method, that can be combined with the example method alone or in combination with other methods, can include the following. Creating the mask includes the following steps. The created secondary projection is normalized. A predetermined threshold is applied to the normalized secondary projection and a binary image is determined as the mask.

Aspects of the example method, that can be combined with the example method alone or in combination with other methods, can include the following. Creating the mask includes creating a shifted mask based on an estimated detector shift between each primary projection and the associated secondary projection.

Aspects of the example method, that can be combined with the example method alone or in combination with other methods, can include the following. Applying the mask includes applying the shifted mask to each interpolated secondary projection to align the isolated ROI with the secondary radiation in the corresponding primary projection.

Aspects of the example method, that can be combined with the example method alone or in combination with other methods, can include the following. Providing the corrected images includes applying, after the subtraction of the associated secondary projection, a median or mean filter configured to remove noise caused by a secondary source associated with the secondary radiation.

Aspects of the example method, that can be combined with the example method alone or in combination with other methods, can include the following. Providing the corrected images includes displaying the corrected images at a display device. Each corrected image is visualized sequentially, or as part of a composite image set in addition to the first data set.

### BRIEF DESCRIPTION OF DRAWINGS

These and other features will be more readily understood from the following detailed description taken in conjunction with the accompanying drawings.

FIG. 1 is a flowchart of an example method that can be used with aspects of this disclosure;

FIG. 2 is a primary projection according to one exemplary embodiment;

FIG. 3 is a secondary projection according to one exemplary embodiment;

FIG. 4 is a mask according to one exemplary embodiment; and

FIG. 5 is the secondary project with an application of the mask.

FIG. 6 is a corrected image with secondary radiation removed after the subtraction of the secondary projection from the primary projection according to one exemplary embodiment;

FIG. 7 is an example imaging processing system that can be used with aspects of this disclosure; and

FIG. 8 is a block diagram of an example controller that can be used with aspects of this disclosure.

### DETAILED DESCRIPTION

Certain implementations will now be described to provide an overall understanding of the principles of the structure, function, manufacture, and use of the devices and methods disclosed herein. One or more examples of these implementations are illustrated in the accompanying drawings. Those skilled in the art will understand that the devices and methods specifically described herein and illustrated in the accompanying drawings are non-limiting implementations and that the scope of the present invention is defined solely by the claims. The features illustrated or described in connection with one implementation may be combined with the features of other implementations. Such modifications and variations are intended to be included within the scope of the present invention.

Further, in the present disclosure, like-named components of the implementations generally have similar features, and thus within a particular implementation each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

In the field of X-ray imaging capturing high-quality images is crucial for accurate geological and equipment assessment. Conventional imaging system often struggle with artifacts caused by backscatter or secondary radiation, commonly known as “off-focus radiation”. These artifacts are captured as low-frequency features which can obscure details and degrade the overall image quality. Such problems are exacerbated in the challenging X-ray imaging sector where precision is paramount for making informed decisions about manufacturing quality, defect recognition and internal structures of multiple subjects.

This disclosure relates to removing these artifacts. Additional projections that include only secondary or backscatter radiation, without the primary radiation signal, are captured and received. By receiving these distinct sets of primary and secondary projections, imaging processing techniques can be used to efficiently and effectively isolate and subtract the secondary radiation from the primary projections, thereby removing the unwanted artifacts, enhancing the charity and utility of the primary projections.

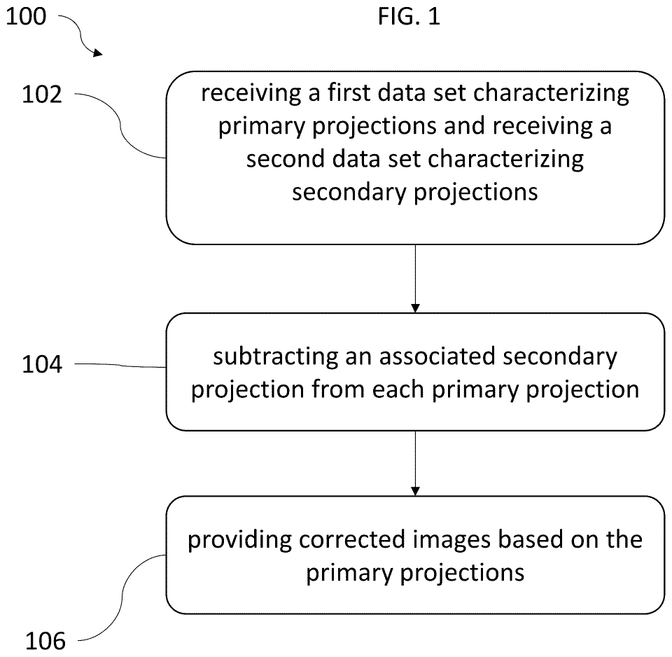

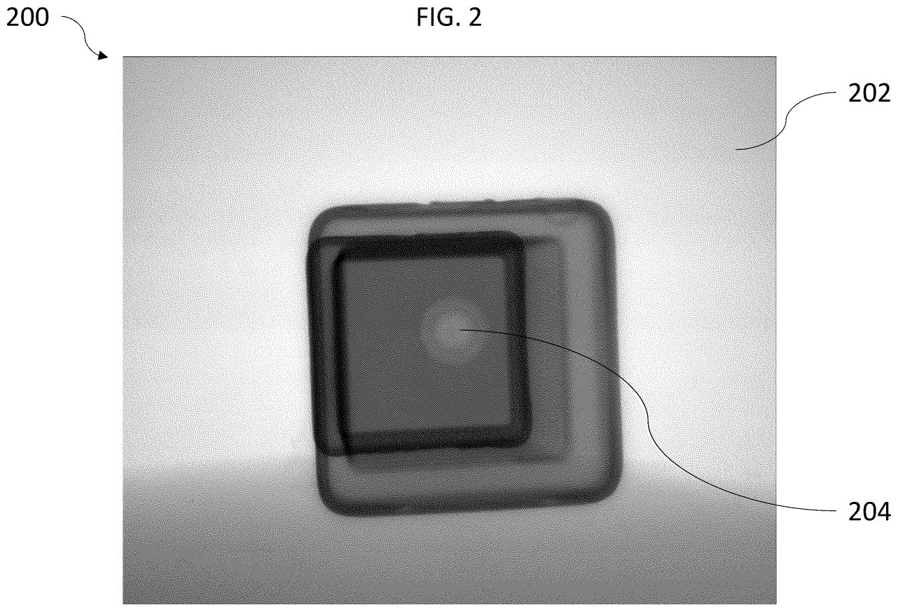

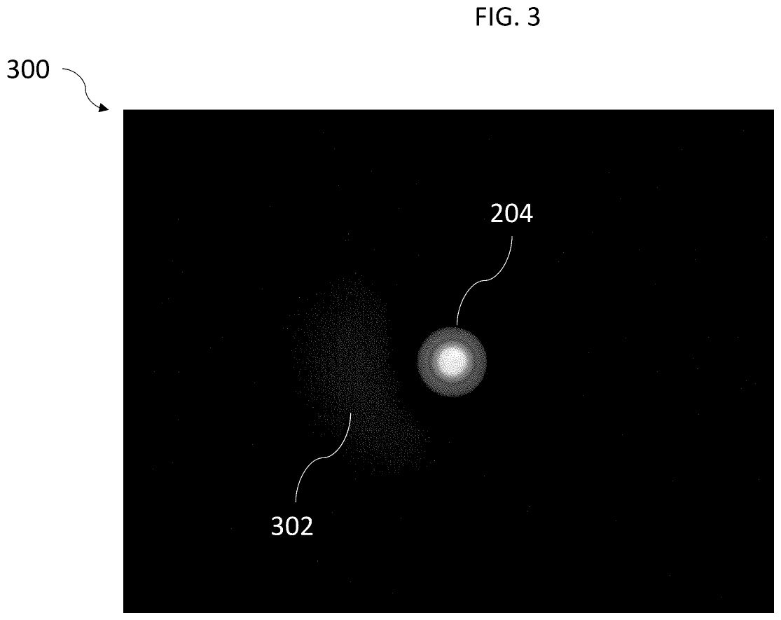

FIG. 1 is a flow chart of an example method 100 that can be used with aspects of this disclosure. At 102, data is received. The data can include information characterizing at least two projections, such as the primary projection 200 shown in FIG. 2 and the secondary projection 300 shown in FIG. 3. In some implementations, projections are captured using an imaging device, such as, an X-ray tube as described throughout this disclosure. The primary projection generates, in this case, a conical-shaped beam (or in some cases, fan-shaped beam). X-ray imaging can include both desired diagnostic radiation and undesired secondary radiation. The diagnostic radiation is the “primary radiation” that is intentionally directed through an object to capture, for instances, structural or compositional information for analysis. The secondary projection, on the other hand, exclusively includes the undesired secondary radiation inadvertently produced due to backscattering processes within the subject or the components of the imaging device itself.

Generally, the X-ray tube is designed to emit radiation that is both polychromatic and directional. The X-ray tube includes an anode, and a cathode enclosed in a vacuum environment e.g., a ceramic or metal enclosure. The cathode is responsible for emitting electrons when heated, and the anode serves as a target material on which the electrons are impinged to produce X-ray beams. In some implementations, a focusing cup can be placed around the cathode and is configured to focus the emitted elections into a narrow beam directed towards the anode in a small area of the material. This highly focused electron beam creates a significant amount of heat in the target. In some implementations, anode may be a rotating anode which, in some cases, help dissipate heat more effectively compared to a stationary anode, allowing for higher intensity and longer imaging sessions without damaging the anode. For this configuration, the anode is manufactured with highly heat-conductive materials that removes the heat away from the focused target area without requiring moving targets.

The backscattering process typically occurs when electrons, after striking the anode, scatter and generate secondary X-rays that are not part of the primary beam path. These X-rays are produced from interactions between the high-energy electrons and the tube enclosure. In some instances, secondary radiation is captured as a less defined mark (e.g., a broad, diffuse, circular pattern as illustrate in FIG. 2) compared to the sharp focus of the primary radiation images in the background. This occurs because secondary radiation includes X-rays that have been created in random places inside the enclosure by the backscattered electrons. To this end, unlike primary radiation that travels directly from the point-like source in the target to the detector, secondary X-rays not just along the intended path of the primary radiation but are emitted in multiple directions due to their scattered origins, resulting in manifestation of noise or artifacts in the final projection.

In some cases, the X-ray tube can have various configurations or operational modes. In some implementations, the X-ray tube is equipped with a “regular scanning mode” (or standard mode) which configure the X-ray tube to emit a continuous beam of X-rays that pass through the subject to capture, for example, one or more primary projections. In some implementations, the X-ray tube also has a specialized “backscatter-only mode” which configure the X-ray tube to primarily emit secondary radiation inside the head portion of the tube and the system can capture, for example, one or more secondary projections. Other operational modes can be incorporated, and the X-ray tube can further switch between these modes as required by the method described herein. In some cases, when switching between the “regular scanning mode” and the “backscatter-only mode”, the anode focusing point, the filament current, voltage settings, and other related operational parameters can be specifically calibrated to enhance (or diminish) the sensitivity to backscatter radiation.

For example, as previously mentioned, FIG. 2 is an image of a primary projection 200 obtained using X-ray tube in the “regular scanning mode”, demonstrating a typical diagnostic projection, where both the desired primary radiation 202 and the undesired secondary radiation 204 are captured. As illustrated in FIG. 2, sharp details of a capacitor are provided by the primary radiation 202. However, the image artifact, provided by the secondary radiation 204 appears in proximity to a center of the primary projection 200 as a circular, diffuse haze that blurs and obscure the details of at least a portion of the object. This is because the object is penetrated by both primary and secondary radiation sources and therefore appear in the same projection.

FIG. 3 is an image of a secondary projection 300 captured using the “backscatter-only mode” of the X-ray tube, which detects and records secondary radiation 204. The secondary projection 300 is captured with object removed from the field of view of the X-ray tube. According to some implementations, secondary projection 300 distinctly displays the characteristics of backscatter radiation as a “bright spot” representative of a centralized, less defined, luminous artifact against a dark background. The bright spot shape is due to the fact that for this instance the enclosure's geometry with it's generatrix normal to the detector. The projection is then the truncation of this tubular X-ray source. Additionally, secondary projection 300 can include noise 302 due to the low power nature of the secondary radiation source.

Referring back to FIG. 1, in some implementations the X-ray tube can switch between the operational modes rapidly, minimizing temporal discrepancies between the two projections. For example, primary projection and secondary projection are captured in quick succession, allowing the image data from both projections are closely synchronized.

In some implementations, projections may be captured sequentially, in an alternating manner. In such cases, projections may be captured one at a time. It should be noted that it is possible to first capture the secondary projection using the X-ray tube in the “backscatter-only mode” and subsequently capture the primary projection using the X-ray tube in the “regular scanning mode”, or vice versa. Such sequence flexibility does not impact the integrity or usability of the captured projections, as both the primary and the secondary projections are independently useful in processing steps as described in further detail within this disclosure.

In some cases, data characterizing primary and secondary projections are loaded from a data storage to a processing or control system. For example, data storage medium, such as a connected USB drive, local storage system (e.g., hard drives or solid-state drives), cloud-based storage, and/or a database that is linked to the system may can be used to archive data characterizing both primary and secondary projections. data stored in these mediums can be systematically indexed based on various parameters such as the date of capture, type of projection (e.g., primary, or secondary), object being imaged, or other specific settings used during the imaging process, allowing for efficient data retrieval for further processing. Receiving the data can include receiving data characterizing projections that are previously captured (e.g., historical scans).

At 104, an associated secondary projection is subtracted from each primary projection. The subtraction involves pixel-by-pixel subtraction of intensity values from two aligned images. In cases where the projections are of different sizes, padding (e.g., a defined amount of space) is applied to the smaller image to ensure that the dimensions of both projections are identical such that the corresponding pixels in both projections are matched. Once the alignment is verified, the intensity value of each pixel in the associated secondary projection is subtracted from the intensity value of the corresponding pixel in the primary projection, which can be performed as follows:

For each pixel located at position (x, y) in the projections, the pixel value in the secondary projection I₂(x, y) is subtracted from the pixel value in the primary projection I₁(x, y).

The resulting pixel value I₃(x, y) is then calculated and stored in a resultant image (e.g., a corrected image as described herein), defined by I₃(x, y)=I₁(x, y)−I₂(x, y).

In some implementations, such subtraction can be performed during the scanning process of each primary projection. Subtraction of the secondary projections for each primary projection can be performed immediately after each scan, without the need to store and process the projections in a separate batch operation. As the primary projection (of a pipeline weld for example) is captured, X-ray tube can simultaneously or immediately after captures the associated secondary projection using the “backscatter-only mode”. During the scanning process, primary and secondary projections are aligned, and the subtraction is performed while the scanning progresses along the length of the weld (e.g., performing the subtraction on-the-fly). Accordingly, corrected images can be produced immediately after each scan.

In some cases, subtraction includes creating a secondary projection with clearest secondary signal measurable (e.g., highest secondary signal-to-noise ratio) to create a mask that is applied across the entire (resampled) second data set. The mask is designed to highlight a region affected by the secondary radiation (e.g., a region of interest) and is configured to, once applied the secondary projections, isolate the region of interest (ROI) for target subtraction (without adversely affecting the overall image quality) as described throughout this disclosure. Such secondary projection is created by setting the system in backscatter-only mode and removing any object between the tube and the X-ray detector. This will allow a clear signal of only the secondary radiation which would also indicate the largest possible “brightest spot” to be captured.

Accordingly, the secondary projection is normalized so that its respective intensity scale is adjusted to have a consistent range of pixel values. In some cases, the normalization can be performed by setting the maximum and minimum intensity values to specific upper and lower bounds, or in other cases, by adjusting the mean and variance of the intensities to a standardized level. A threshold is calculated by means of the triangle method to create a binary image. The pixels of the binary image are assigned a value of 1 (white) if their intensity is above the threshold and a value of 0 (black) otherwise. In this case, the pixels having value of 1, in combination, defines the ROI that is affected by the secondary radiation. The background information is represented by pixels having value of 0.

In the binary image, the bright spot is characterized as a connected pixel region (or a contiguous area) having pixel values of 1s. The connected pixel region may include a cluster of adjacent pixels that share the same binary value (e.g., value of 1). In some implementations, one or more mathematical morphology operation and/or imaging processing algorithms, such as image dilation and connected component analysis (CCA) can be performed, in cases where multiple connected pixel regions are presented, to effectively identify a connected pixel region having a largest area or a highest pixel count. CCA, in some instances, can search for connected pixel regions via 4-connectivity (considering up, down, left, and right) or 8-connectivity (which includes diagonal neighbors as well).

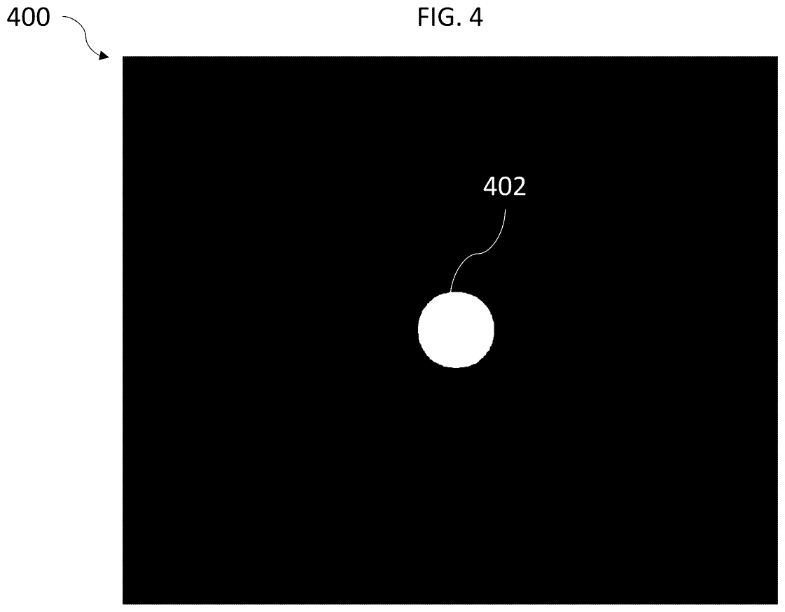

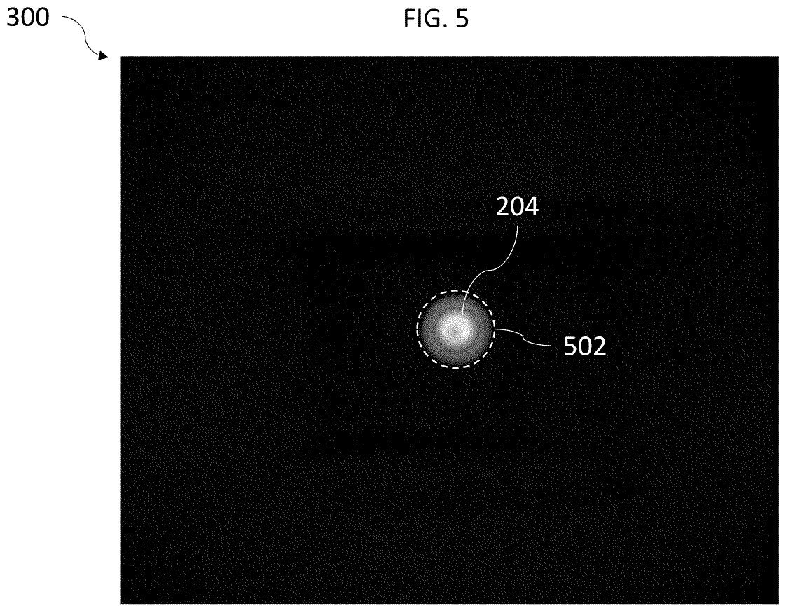

The mask, in certain implementations, can be the binary image created from the secondary projection with highest secondary signal-to-noise ratio. For example, as shown in FIG. 4, the mask 400 includes the ROI 402 (containing pixels with a value of 1) that represents the area of highest impact from secondary radiation against a black background (containing pixels with a value of 0). As shown in FIG. 5, when the mask 400 is applied to each secondary projection within the second data set (e.g., by multiplying their pixel values), it isolates the specific areas e.g., area 502 where the artifacts are most prominent, ensuring that only the isolated area 502 represented by the white pixels in the mask 400 are considered for the subtraction, preserving the details of the surrounding areas in the secondary projection 300.

In some cases, one or more filtering techniques can be applied to the secondary projection to create the mask 400. For example, a median filter may be applied to the secondary projection 300 to remove the noise 302. All the pixel values from a pre-defined, surrounding neighborhood (e.g., an array of pixel values with a length equal to the number of pixels within the pre-defined, surrounding neighborhood) are sorted into numerical order, and the pixel being considered (at each iteration) is being replaced with a median value from the array, effectively removing “salt and pepper” noises 302 while preserving the edges of the secondary radiation 204. The size of the filter depends on the extent of noise and the desired level of detail preservation. Typically, a median filter of size 3×3 or 5×5 pixels is used.

The mask also reduces the processing time of the direct subtraction. Full-image subtraction (without the mask) involves pixel-by-pixel processing across the entire image. According to some implementations, for images of size n×n, each pixel from the secondary projection may need to be subtracted from the primary projection, leading to a complexity of O(n²), which can be significantly higher than the localized-image subtraction (with the mask), especially for large images, since the latter only processes pixels within the mask's boundaries, and is therefore limited to the region defined by the mask. Direct subtraction, in this case, subtracts only the ROI 402 of the secondary projection to ensure that the subtraction process effectively removes secondary radiation artifacts without impacting surrounding image areas in the corresponding primary projection that are free of such artifact.

In some cases, the mask 400 are shifted to compensate for positional discrepancy (raised during the interpolation process) between the original secondary projection and the interpolated secondary projection. The detector shift between the primary projection and its associated secondary projection can be estimated based on known movement parameters of the imaging system. For example, when the detector is moved by a few pixels to reduce the impact of a damaged pixel in the detector. The shifted mask is then applied to the interpolated secondary projections to compensate geometric distortions caused by the interpolation process, ensuring accurate artifact correction.

At 106, the corrected images can be provided subsequent to the subtraction process described herein. The corrected images, in some implementations, represent the object being imaged with improved clarity, as the noise and artifacts caused by the backscattered or secondary radiation are no longer present. For example, the corrected images maintain the details captured by the primary projections, while the diffuse or blurred regions (which are the result of the secondary radiation interference) are eliminated.

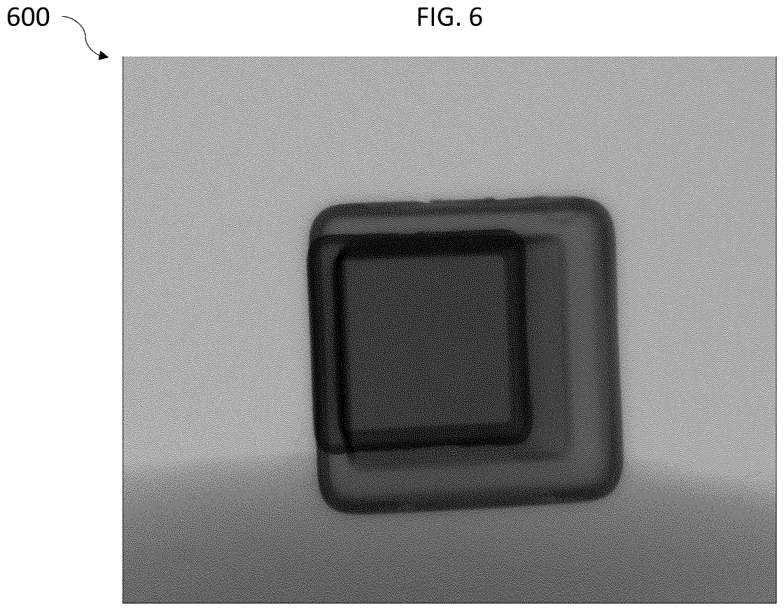

For example, FIG. 6 is an exemplary embodiment of a corrected image 600 obtained after the direct subtraction of the processed secondary projection 500 from the primary projection 200 as described above with reference to FIGS. 2-4. As shown in FIG. 6, the corrected image 600 presents a clearer image where the effects or secondary radiation 204 have been removed sufficiently for the corrected image to be used for analysis. That is, the capacitor appears with enhanced clarity compared to FIG. 2, as the bright spot at the center of the image has been sufficiently removed.

The corrected images may be subjected to additional processing steps. In some cases, one or more filtering techniques can be applied to further enhance the quality of the projections. For example, the median filter may be applied to each corrected image (e.g., primary projection post subtraction) to remove residual noise. It should be noted that the selection of the appropriate filter depends on the specific system requirements and the characteristics of the noise or artifacts to be addressed. Other filtering techniques, such as Gaussian filters, Bilateral filters, Wiener filters, Sobel filters, and the like can be used to improve the visual quality of the projections at any step throughout method 100, prior to the method 100, and/or after the method 100 as described herein. In some cases, these filtering techniques can be applied individually or in combination where the strengths of one filter compensate for the limitations of another. For instance, combing a Gaussian filter with the median filter can help effectively reduce both Gaussian noise and the “salt-and-pepper” noise in the projections.

Providing the corrected images can include displaying the corrected images for inspection, storing the corrected images in a data storage system, and/or transmitting the corrected images to one or more downstream devices (or connected network) for further analysis. For example, the method 100 as described herein can be a part of an industrial quality control process where the corrected images can be used by technicians to assess the integrity of components, such as detecting cracks, voids, or material inconsistencies in the structures. Alternatively, or in addition, the corrected images can be compared against one or more baseline or historical scan for monitoring, for example the progression of material degradation over time. In some implementations, the corrected images (captured at different angles) can be used as inputs for computed tomography (CT) reconstruction, enabling three-dimensional (3D) imaging of the object's internal structure for enhanced analysis and defect detection.

The corrected images can be displayed at a display device, such as, without limitation, a high-resolution monitor, a handheld device with a display screen built-in, or a specialized industrial imaging station. In some implementations, the corrected images can be visualized sequentially; for example, a series of corrected images can be displayed in a slideshow format to observe changes or improvements over a sequence of scans. In some cases, a composite image set can be provided where different images, such as primary projections before and after the correction, are displayed simultaneously. This can include, for example, and without limitation, side-by-side views or overlay views (e.g., superimposing the corrected image over the original primary projection or vice versa with varying opacity) for comparison between the original and the corrected images and better illustration of the comparison result.

However, in situation involving rapid, high-volume inspection process (e.g., where dynamic changes in the object being imaged are expected), there is a need to capture a large number of (primary) projections (e.g., 10, 50, 100, 200, 500 or more) in a quick succession. Therefore, an equivalent number of secondary projections is typically captured to match each primary projection. This introduces challenges related to the time and resources required for data acquisition, since the subtraction of the secondary radiation from the primary projection requires at least one corresponding secondary projection for each primary projection, doubling the total acquisition time.

To reduce the number of secondary projections needed, data characterizing fewer (e.g., one secondary projection for every 100 primary projections) is received. Such data is then resampled via an interpolation technique. The image interpolation can be used to estimate unknown pixel based on known values of the surrounding pixels. For instance, resampling the second data set includes applying linear interpolation across the second data set according to the following formula:

$$p ( x ) = f ( {x}_{0} ) + \frac{f ( {x}_{1} ) - f ( {x}_{0} )}{{x}_{1} - {x}_{0}} ( x - {x}_{0} )$$

Where f(x₀) and f(x₁) are the intensity values of the secondary radiation at known angles x₀ and x₁ respectively, p(x) is the predicted intensity value at a new angle x, which is not directly captured but is within the range between x₀ and x₁. The formula is applied on a pixel-by-pixel basis across the second data set. Accordingly, a set of intermediate secondary projections that do not need to be physically captured can be provided. The set of intermediate secondary projections can be used just like the original secondary projections to subtract from the primary projections, thus effectively removing the artifacts associated with the secondary radiation in less time and reducing the computational load.

Each pixel's intensity in the interpolated image can be calculated based on its corresponding pixels in the secondary projections captured at angles x₀ and x₁. This interval may be pre-selected based on the level of detail needed or the eristic measurements in the corrected images. In some implementations, the secondary projections within the second data set are captured at every 36 degrees from 0 to 360 degrees. For example, a first secondary projection and a second secondary projection are captured at 0 degrees and 180 degrees respectively. The interpolated images may represent estimated secondary projections (e.g., 100 projections in total) at an angle not directly captured but calculated to be between 0 degrees and 180 degrees. The interpolated images are then added back to the second data set to increase the effective sample size of the secondary projections.

It should be noted that other interpolation techniques, such as quadratic interpolation, sinogram interpolation, spline interpolation, and the like can also be employed depending on the desired level of performance and the specific requirements of the imaging task. In some implementations, the total number of projections (typically 1000 per data set) should be divisible by the sample distance to ensure that each interpolated projection is spaced uniformly across the imaging spectrum. To evaluate the effectiveness of different interpolation techniques, metrics such as the mean square error (MSE) between the interpolated secondary projection p and the original secondary projection f are typically used. The MSE is calculated as follows:

$$M S E = {\sum }_{i = 1}^{I} {( p ( {x}_{i} ) - f ( {x}_{i} ) )}^{2}$$

Where I is the number of pixels in one image. MSE quantifies the average of the squares of the errors—the average squared difference between the estimated values and what is estimated. For example, a lower MSE indicates a higher accuracy of the interpolation in preserving the details and characteristics of the original projection.

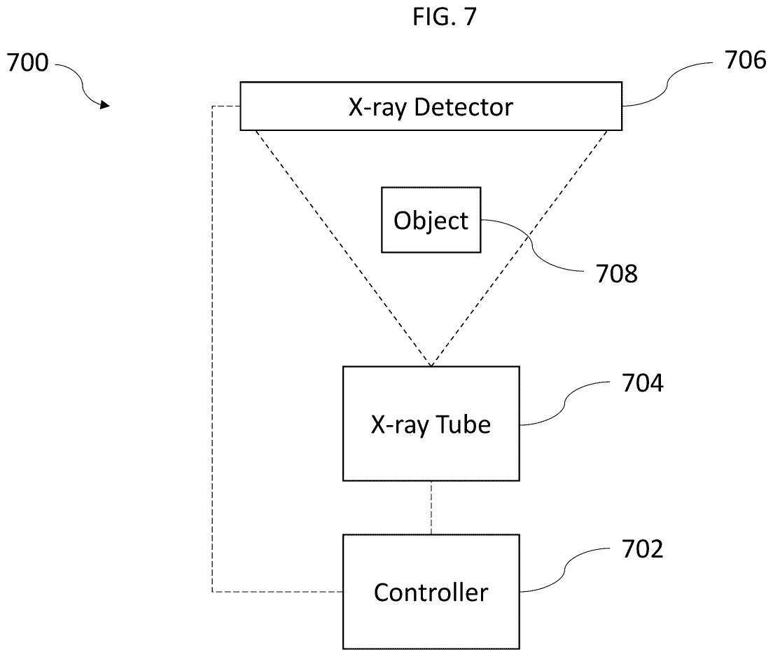

FIG. 7 illustrates a block diagram of the image processing system 700 designed to remove artifacts caused by the secondary radiation. The image processing system 700 includes at least one controller 702 capable of executing one or more processing steps or any combinations thereof as described above with reference to FIGS. 1-5. The controller 702 is configured to receive data characterizing multiple primary projections and data characterizing multiple secondary projections. These projections are captured using an X-ray imaging system which includes an X-ray tube 704 and an X-ray detector 706. Generally, the X-ray tube 704 emits radiation e.g., both primary radiation 708 and secondary radiation 710, that passes through an object 712, and the X-ray detector 706 captures the resulting image of the object 712. The captured images are inputted into the image processing system 700 through one or more data interfaces that may include wired or wireless communication channels.

As described previously, the primary radiation 708 is generated when electrons emitted from the cathode are accelerated and strike towards the anode at a focal spot within the X-ray tube 704. The primary radiation 708 follows a conical path from the focal spot through the tube's output port. For example, as shown in FIG. 7, the primary radiation 708 is conical or fan shaped as it spreads out from the focal spot. This conical spread is due to the divergence of the X-ray beam as it exits the focal spot. However, not all electrons contribute to the primary radiation 708. The secondary radiation 710, on the other hand, originates from interactions involving backscattered electrons and the vacuum enclosure. The primary radiation 708 can cover a larger area of the target object compared to the secondary radiation 710.

In some implementations, the controller 702, in conformance with the data received from the X-ray imaging system or user inputs, controls the operational modes of the X-ray tube 704. The X-ray tube 704 and the X-ray detector 706 are in substantially diametric opposition to one another. In some cases, the controller 702 can cause the X-ray tube 704 and the X-ray detector 706 to rotate about the object 712 and vice versa, move the object 712 about a fixed X-ray tube 704 and the X-ray detector 706. The X-ray tube 704 can emit radiation in a cone shape (or a fan shape) to which at least a portion of the object 712 is exposed. The radiation is subsequently absorbed by the X-ray detector 706 and the corresponding projection is constructed via signal conversion.

The controller 702 can process the projections using one or more image processing algorithms as described herein. with both data characterizing primary projections and data characterizing secondary projections at its disposal, the image processing system 700 executes the subtraction algorithm to remove corresponding secondary radiation artifact presented in each secondary projection from each primary projection. To improve the overall efficiency of the image processing system 700, each secondary projection is captured at a predetermined sample distance and the data characterizing secondary projections is further resampled via interpolation techniques to increase the effective sample size of the secondary projections, reducing the scanning times during the secondary projections acquisition. Additionally, each secondary projection is masked to ensure that only the unwanted radiation is removed without affecting the desired details of the primary projections.

The controller 702 is capable of outputting, upon the execution of the subtraction algorithm, processed projections, such as a set of corrected images as described throughout this disclosure. Each corrected image represents a primary projection with the secondary radiation artifacts removed. In some cases, the controller 702 can output the set of corrected images to one or more downstream devices or connected systems or networks via the data interfaces. In some implementations, the imaging processing system 700 further includes a display device communicatively connected to the controller 702 configured to display part or all of the set of corrected images for review or further analysis in digital formats. We should definitely mention tomography here too. The images are transformed to tomographic data.

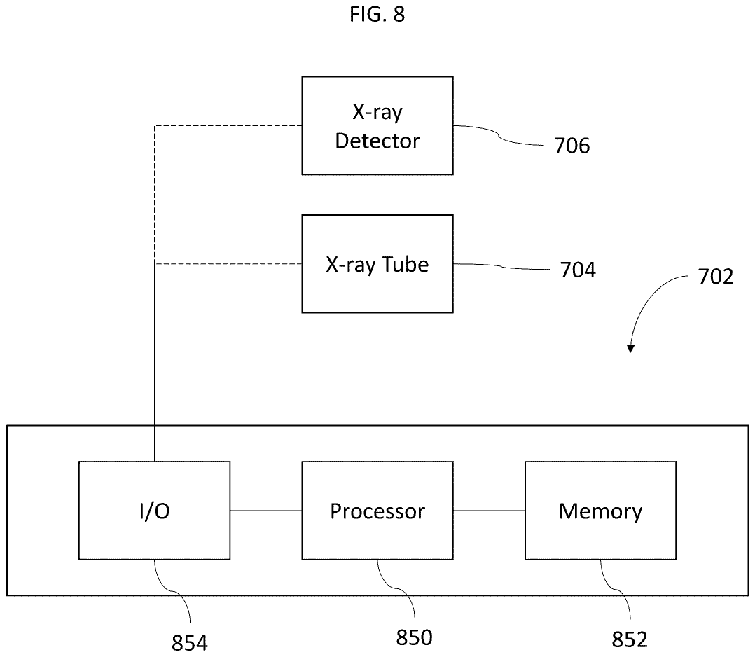

An example controller is illustrated in FIG. 8. In some implementations, the controller 702 can execute all or part of the method 100. The controller 702 can, among other things, manage the configuration of the X-ray imaging system. As shown in FIG. 8, the controller 800 can include one or more processors 850 and non-transitory computer readable memory storage (e.g., memory 852) containing instructions that cause the processors 850 to perform operations. The processors 850 are coupled to an input/output (I/O) interface 854 for sending and receiving communications with components in the image processing system 700, including, for example, the X-ray tube 704 and the X-ray detector 706. In certain instances, the controller 702 can additionally communicate status with and send actuation and/or control signals to one or more of the various system components of the system. Other aspects of the method 100 can similarly be performed by the controller with various degrees of autonomy, for example, adjusting one or more processing parameters or variables used herein based on feedback from the evaluations for specified outputs. Alternatively, or in addition, a user can interact with the system described herein to manually customize processing parameters or input specific commands for additional processing.

Certain exemplary implementations will now be described to provide an overall understanding of the principles of the structure, function, manufacture, and use of the systems, devices, and methods disclosed herein. One or more examples of these implementations are illustrated in the accompanying drawings. Those skilled in the art will understand that the systems, devices, and methods specifically described herein and illustrated in the accompanying drawings are non-limiting exemplary implementations and that the scope of the present invention is defined solely by the claims. The features illustrated or described in connection with one exemplary implementation may be combined with the features of other implementations. Such modifications and variations are intended to be included within the scope of the present invention. Further, in the present disclosure, like-named components of the implementations generally have similar features, and thus within a particular implementation each feature of each like-named component is not necessarily fully elaborated upon.

The subject matter described herein can be implemented in digital electronic circuitry, or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine-readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processor of any kind of digital computer. Generally, a processor will receive instructions and data from a Read-Only Memory or a Random Access Memory or both. The essential elements of a computer are a processor for executing instructions and one or more memory devices for storing instructions and data. Generally, a computer will also include or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having a display device, e.g., a CRT (cathode ray tube) or LCD (liquid crystal display) monitor, for displaying information to the user and a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The techniques described herein can be implemented using one or more modules. As used herein, the term “module” refers to computing software, firmware, hardware, and/or various combinations thereof. At a minimum, however, modules are not to be interpreted as software that is not implemented on hardware, firmware, or recorded on a non-transitory processor readable recordable storage medium (i.e., modules are not software per se). Indeed “module” is to be interpreted to always include at least some physical, non-transitory hardware such as a part of a processor or computer. Two different modules can share the same physical hardware (e.g., two different modules can use the same processor and network interface). The modules described herein can be combined, integrated, separated, and/or duplicated to support various applications. Also, a function described herein as being performed at a particular module can be performed at one or more other modules and/or by one or more other devices instead of or in addition to the function performed at the particular module. Further, the modules can be implemented across multiple devices and/or other components local or remote to one another. Additionally, the modules can be moved from one device and added to another device, and/or can be included in both devices.

The subject matter described herein can be implemented in a computing system that includes a back-end component (e.g., a data server), a middleware component (e.g., an application server), or a front-end component (e.g., a client computer having a graphical user interface or a web interface through which a user can interact with an implementation of the subject matter described herein), or any combination of such back-end, middleware, and front-end components. The components of the system can be interconnected by any form or medium of digital data communication, e.g., a communication network. Examples of communication networks include a local area network (“LAN”) and a wide area network (“WAN”), e.g., the Internet.

Approximating language, as used herein throughout the specification and claims, may be applied to modify any quantitative representation that could permissibly vary without resulting in a change in the basic function to which it is related. Accordingly, a value modified by a term or terms, such as “about” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

What is claimed is:

1. A method comprising:
receiving a first data set characterizing a plurality of primary projections, wherein each one of the plurality of primary projections includes both primary radiation and secondary radiation;
receiving a second data set characterizing a plurality of secondary projections, wherein each one of the plurality of secondary projections includes only secondary radiation;
subtracting an associated secondary projection from each primary projection of the plurality of primary projections; and
providing a plurality of corrected images with the secondary radiation removed in each one of the plurality of corrected images based on the plurality of primary projections.

1. The method of claim 1, wherein the plurality of primary projections is captured using an X-ray tube in a first operational mode, wherein the plurality of secondary projections is captured using the X-ray tube in a second operational mode.

2. The method of claim 1, further comprising resampling the second data set by interpolating between the plurality of secondary projections, wherein resampling comprises:
determining an angular interval between each secondary projection of the plurality of secondary projections;
interpolating between the plurality of secondary projections at the determined angular interval to generate a plurality of interpolated secondary projections; and
adding the plurality of interpolated secondary projections to the second data set.

3. The method of claim 3, wherein the angular interval is determined to range from 0 to 180 degrees.

4. The method of claim 1, wherein the subtracting comprises:
creating a secondary projection with a highest secondary signal-to-noise ratio;
creating a mask based on the created secondary projection;
applying the mask to each secondary projection of the plurality of secondary projections to isolate a region of interest (ROI) affected by the secondary radiation; and
subtracting the ROI from each primary projection of the plurality of primary projections.

5. The method of claim 5, wherein creating the mask comprises:
normalizing the created secondary projection; and
applying a predetermined threshold to the normalized secondary projection to determine a binary image.

6. The method of claim 5, wherein creating the mask further comprises:
creating a shifted mask based on an estimated detector shift between each primary projection of the plurality of primary projections and the associated secondary projection.

7. The method of claim 7, wherein applying the mask to each secondary projection of the plurality of secondary projections comprises:
applying the shifted mask to each interpolated secondary projection of the plurality of secondary projections to align the isolated ROI with the secondary radiation in the corresponding primary projection.

8. The method of claim 1, wherein providing the plurality of corrected images comprises:
applying, for each secondary projection of the plurality of secondary projections, a median filter configured to remove noise caused by a secondary source associated with the secondary radiation.

9. The method of claim 1, wherein providing a plurality of corrected images further comprises:
displaying, at a display device, the plurality of corrected images, wherein each corrected image of the plurality of corrected images is visualized sequentially, or as part of a composite image set in addition to the plurality of primary projections.

10. A system comprising:
at least one data processor; and
non-transitory memory storing instructions, which, when executed by the at least one data processor causes the at least one data processor to perform operations comprising:
receiving a first data set characterizing a plurality of primary projections, wherein each one of the plurality of primary projections includes both primary radiation and secondary radiation;
receiving a second data set characterizing a plurality of secondary projections, wherein each one of the plurality of secondary projections includes only secondary radiation;
subtracting an associated secondary projection from each primary projection of the plurality of primary projections; and
providing a plurality of corrected images with the secondary radiation removed in each one of the plurality of corrected images based on the plurality of primary projections.

11. The system of claim 11, wherein the plurality of primary projections is captured using an X-ray tube in a first operational mode, wherein the plurality of secondary projections is captured using the X-ray tube in a second operational mode.

12. The system of claim 11, wherein the operations further comprising resampling the second data set by interpolating between the plurality of secondary projections, wherein resampling comprises:
determining an angular interval between each secondary projection of the plurality of secondary projections;
interpolating between the plurality of secondary projections at the determined angular interval to generate a plurality of interpolated secondary projections; and
adding the plurality of interpolated secondary projections to the second data set.

13. The system of claim 13, wherein the angular interval is determined to range from 0 to 180 degrees.

14. The system of claim 11, wherein the subtracting comprises:
creating a secondary projection with a highest secondary signal-to-noise ratio;
creating a mask based on the created secondary projection;
applying the mask to each secondary projection of the plurality of secondary projections to isolate a region of interest (ROI) affected by the secondary radiation; and
subtracting the ROI from each primary projection of the plurality of primary projections.

15. The system of claim 15, wherein creating the mask comprises:
normalizing the created secondary projection; and
applying a predetermined threshold to the normalized secondary projection to determine a binary image.

16. The system of claim 15, wherein creating the mask further comprises:
creating a shifted mask based on an estimated detector shift between each primary projection of the plurality of primary projections and the associated secondary projection.

17. The system of claim 17, wherein applying the mask to each secondary projection of the plurality of secondary projections comprises:
applying the shifted mask to each interpolated secondary projection of the plurality of secondary projections to align the isolated ROI with the secondary radiation in the corresponding primary projection.

18. The system of claim 11, wherein providing the plurality of corrected images comprises:
applying, for each secondary projection of the plurality of secondary projections, a median filter configured to remove noise caused by a secondary source associated with the secondary radiation.

19. A non-transitory computer readable memory storing instructions which, when executed by at least one data processor forming part of at least one computing system, causes the at least one data processor to perform operations comprising:
receiving a first data set characterizing a plurality of primary projections, wherein each one of the plurality of primary projections includes both primary radiation and secondary radiation;
receiving a second data set characterizing a plurality of secondary projections, wherein each one of the plurality of secondary projections includes only secondary radiation;
subtracting an associated secondary projection from each primary projection of the plurality of primary projections; and
providing a plurality of corrected images with the secondary radiation removed in each one of the plurality of corrected images based on the plurality of primary projections.
